SEQUENCE LISTING (8 sequences)

SEQ ID NO: 1 - DNA, 1155 bp, Artificial
  mutant human APOBEC3G
atgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacgacgattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactga

SEQ ID NO: 2 - protein, 383 aa, Artificial
  mutant human APOBEC3G
MKPHFRNTVEMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDGDDQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQQN

SEQ ID NO: 3 - DNA, 1155 bp, Artificial
  APOBEC3G Mutant
atgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacggagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactga

SEQ ID NO: 4 - protein, 383 aa, Artificial
  mutant human APOBEC3G
MKPHFRNTVEMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDGDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQQN

SEQ ID NO: 5 - DNA, 1155 bp, Artificial
  APOBEC3G Mutant
atgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacgcagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactga

SEQ ID NO: 6 - protein, 383 aa, Artificial
  mutant human APOBEC3G
MKPHFRNTVEMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDADYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQQN

SEQ ID NO: 7 - DNA, 1155 bp, Artificial
  mutant human APOBEC3G
atgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactattcctgggacgacgattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactga

SEQ ID NO: 8 - protein, 383 aa, Artificial
  mutant human APOBEC3G
MKPHFRNTVEMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDGDFQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQQN

(19) United States
(12) Patent Application Publication
(10) Pub. No.: US 2008/0026982 A1
(43) Pub. Date: Jan. 31, 2008

(54) Mutant APOBEC3G molecules for inhibiting replication of HIV

(76) Inventor: Hongzhan Xu, Frederick, MD (US)

Correspondence Address: KALIKO & YEAGER, L.L.C., 500 NORTH FRANKLIN TURNPIKE, RAMSEY, NJ, 07446, US

(21) Appl. No.: 11/489,692

(22) Filed: Jul. 19, 2006

(51) Int. Cl. A61K 38/00; C07H 21/04; C07K 14/00; C07K 16/18; C12N 15/00; C12N 5/06

(52) U.S. Cl. 514/2; 435/320.1; 435/325; 530/350; 530/387.1; 536/23.1

(57) ABSTRACT

Provided herein are novel APOBEC3G polypeptides having an amino acid substitution at position 129, e.g., P129D in which the polypeptide is capable of resisting proteosomal degradation induced by HIV-Vif. Also provided are isolated nucleic acids encoding the APOBEC3G polypeptides, recombinant vectors comprising the nucleic acids, recombinant host cells comprising the nucleic acid molecules encoding the polypeptides, pharmaceutical composition comprising the encoded protein, methods for resisting proteosomal degradation induced by HIV-Vif and thereby reducing or inhibiting HIV-1 and/or HIV-2 infection.

### FIELD OF THE INVENTION

This invention relates to novel polypeptides and methods, which are particularly useful for inhibiting HIV-1 and/or HIV-2 replication.

### BACKGROUND OF THE INVENTION

Apolipoprotein B mRNA-editing enzyme-catalytic polypeptide-like 3G (APOBEC3G), first identified as CEM15, is a host cellular protein with a broad antiviral activity. It inhibits infectivity of a wide variety of retroviruses by deaminating deoxycytidine (dC) into deoxyuridine (dU) in newly synthesized minus strand DNA, resulting in G-to-A hypermutation of the viral plus strand DNA. Harris et al. Cell. 113(6), 803-809 (2003).

The APOBEC3G gene is a member of the cytidine deaminase gene family. It is one of seven related genes or pseudogenes found in a cluster, thought to result from gene duplication, on chromosome 22. It is thought that the proteins may be RNA editing enzymes and have roles in growth or cell cycle control. The protein encoded by this gene has been found to be a specific inhibitor of human immunodeficiency virus-1 (HIV-1 ) and some simian immunodeficiency viruses infectivities in the absence of their viral infectivity factors (Vifs).

It is known that the HIV-1 Vif protects viral replication from a host restriction factor that induces hypermutation of the HIV-1 genome. Lecossier et al., Science 300, 1112 (2003); Mangeat et al., Nature 424, 99-103 (2003); Zhang et al., Nature 424, 94-98 (2003); Harris et al., Cell 113, 803-809(2003). Virion infectivity factor (Vif) binds to APOBEC3G and induces its rapid degradation, thus eliminating it from cells and preventing its incorporation into HIV-1 virions. Vif contains two domains, one that binds APOBEC3G and another with a conserved SLQ(Y/F)LA motif that mediates APOBEC3G degradation by a proteasome-dependent pathway. Kabat et al., U.S. Pat. Publ. No. 2004/0234956.

Recently, it was shown that a single amino acid change at position 128 of human and African green monkey APOBEC3G governs the virus-specific sensitivity of these proteins to Vif-mediated inhibition. Mangeat et al., J. Biol Chem. 2004 Apr. 9;279(15):14481-3. Epub 2004 Feb. 13. Moreover, species specificity of Vif for APOBEC3G was shown to be determined by a single amino acid change at position 128. Schrofelbauer et al., Proc Natl Acad. Sci USA. 2004 Mar. 16;101(11):3927-32. Epub 2004 Feb. 20. While these and other studies focus on the exchange at position 128, efforts to deduce binding or inhibition of APOBEC3G by mutating other amino acid to resist degradation has not been elucidated.

Much is known about Vif, which binds to APOBEC3G and triggers its polyubiquitination and rapid degradation. Navarro and Landau, Curr Opin Immunol. 2004 August;16(4):477-82. Human immune cells possess a built-in mechanism that could potentially block the replication of retroviruses such as HIV-1 . This protective mechanism centers on APOBEC3G, which is incorporated into virions and can ultimately halt completion of the HIV life cycle. However, HIV-1 encodes a protein Vif that specifically suppresses the activity of APOBEC3G. Vif achieves this effect by depleting the intracellular stores of APOBEC3G, thus making this antiviral enzyme unavailable for incorporation into budding virions. APOBEC3G depletion involves the recruitment of a specific E3 ligase complex by Vif leading to the polyubiquitylation and proteasome-mediated degradation of this enzyme. The potent activity of APOBEC3G has led to considerable interest in the identification of small molecules that interrupt the Vif-induced degradative process. Stopak and Greene, Curr Opin Investig Drugs. 2005 February;6(2): 141-7.

Accordingly, a need exists for inhibiting Vif-induced degradative process on APOBEC3G and developing a method for inhibiting replication of HIV. What is needed, therefore, is a APOBEC3G molecule that is resistant to Vif-induced degradation to prevent HIV-1 and/or HIV-2 infection.

### SUMMARY OF THE INVENTION

The present invention provides isolated polypeptides comprising or consisting of polypeptide sequences selected from the group consisting of sequences encoded by the human APOBEC3G having at least one amino acid substitution at position 129 including SEQ ID NOs: 2, 4, 6 and 8 and related polypeptide sequences such as analogs, variants, fragments and fusions thereof that are capable of resistance to proteosomal degradation induced by HIV-Vif.

The invention further provides isolated polynucleotides comprising or consisting of nucleic acid sequences selected from the group consisting of the human APOBEC3G nucleic acids including SEQ ID NO: 1, 3, 5 and 7, related nucleic acid sequences and fragments of mutant APOBEC3G gene; nucleic acid sequences that are degenerate and variants of these sequences. The invention also provides vectors and recombinant host cells comprising these polynucleotides.

In addition, the invention provides methods for expressing the polypeptides, vectors encoding the polypeptides, recombinant host cells comprising the polypeptides, assays for determining viral infectivity and pharmaceutical compositions, e.g., medicaments comprising the proteins. Also provided are methods for treating a subject having HIV-1 and/or HIV-2 infection, for example, by administering an effective amount of the APOBEC3G protein to inhibit HIV replication.

Antibodies that specifically bind to the isolated polypeptides of the invention are also provided.

### BRIEF DESCRIPTION OF THE DRAWINGS

FIG. 1 shows the sequence of APOBEC3G and several mutants including P129F, P129D, P129A and D128K.

FIG. 2 illustrates a plasmid map of APOBEC3G expression vector used to express wild type APOBEC3G protein or its mutant proteins.

FIG. 3 depicts a graph of the results showing anti-retroviral activities of wild type APOBEC3G proteins and its mutants in the absence of HIV Vifs or in the presence of HIV-1 or HIV-2 Vifs.

FIG. 4 depicts an image of a SDS-PAGE and immunoblotting analysis of proteins expressed from APOBEC3G expression vectors and Vif expressing vectors cotransfected into 293T cells. FIG. 4A depicts cellular protein levels of wild type APOBEC3G and its mutants (D128K, P129A, P129D and P129F) in the absence of HIV-1 or HIV-2 Vifs. FIG. 4B shows cellular protein levels of wild type APOBEC3G and its mutants in the presence of HIV-1 Vif. FIG. 4C shows cellular protein levels APOBEC3G and its mutants in the presence of HIV-2 Vif.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2nd ed., Cold Spring Harbor Laboratory Press, Cold Spring Harbor, N.Y. (1989 ); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, a “nucleic acid comprising SEQ ID NO:X” refers to a nucleic acid, at least a portion of which has either (i) the sequence of SEQ ID NO:X, or (ii) a sequence complementary to SEQ ID NO:X. The choice between the two is dictated by the context. For instance, if the nucleic acid is used as a probe, the choice between the two is dictated by the requirement that the probe be complementary to the desired target.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does-not necessarily require that the nucleic acid or polynucleotide so described has itself been physically removed from its native environment. For instance, an endogenous nucleic acid sequence in the genome of an organism is deemed “isolated” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 90%, more preferably 95% of the nucleotide bases, usually at least about 96%, more usually at least about 80%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, intemucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids. The term “nucleic acid” encompasses both DNA and RNA without size limits from any source comprising natural and non-natural bases. Nucleic acids may have a variety of biological functions. They may encode proteins, comprise regulatory regions, function as inhibitors of gene or RNA expression (e.g., antisense DNA or RNA or RNAi), function as inhibitors of proteins, function to inhibit cell growth or kill cells, catalyze reactions, or function in a diagnostic or other analytical assay. Nucleic acids used in preferred embodiments may be in a variety of forms. They may be single stranded, double stranded, branched or modified by the ligation of non-nucleic acid molecules. They may be in a linear form or a closed circle form. In some embodiments, plasmid DNA is used as the nucleic acid. Plasmid DNA is a variety of closed circular DNA and preferably contains a bacterial origin of replication or an equivalent sequence that allows the replication of the DNA molecule in a biological system. RNA interference or “RNAi” is a term initially coined by Fire and co-workers to describe the observation that double-stranded RNA (dsRNA) can block gene expression when it is introduced into worms (Fire et al., Nature 391:806-811, 1998).

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “vector” as used herein is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”). In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art. An exemplary expression vector system is a retroviral vector system such as is generally described in PCT/US97/01019 and PCTUS97/01048, both of which are hereby expressly incorporated by reference. Constructs also are described in U.S. Pat. No. 6,153,380.

As used herein, the term “APOBEC3G” short for apolipoprotein B mRNA-editing enzyme-catalytic polypeptide-like 3G is defined herein as a human protein that interferes with the replication of HIV by incorporating itself into virus particles and damaging the genetic material of the virus. The viral protein Vif can halt this process in two ways: (1) by binding to APOBEC3G and preventing it from incorporating into virus particles; and (2) by targeting APOBEC3G for destruction and almost completely eliminating it from the cell.

The term “marker sequence” or “marker gene” refers to a nucleic acid sequence capable of expressing an activity that allows either positive or negative selection for the presence or absence of the sequence within a host cell. Marker sequences or genes do not necessarily need to display both positive and negative selectability. The expression vector may contain a selectable marker gene to allow the selection of transformed host cells. Selection genes are well known in the art and will vary with the host cell used.

“Operatively linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mNRA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences. In general, the transcriptional and translational regulatory sequences may include, but are not limited to, promoter sequences, ribosomal binding sites, transcriptional start and stop sequences, translational start and stop sequences, and enhancer or activator sequences. In one embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. Recombinant host cells are understood to encompass those cells that have been transfected or transformed. Accordingly, “transfection” used herein generally is understood to mean the delivery and introduction of biologically functional nucleic acid into a cell, e.g., a eukaryotic cell, in such a way that the nucleic acid retains its function within the cell. Transfection encompasses delivery and introduction of expressible nucleic acid into a cell such that the cell is rendered capable of expressing that nucleic acid. The term “expression” means any manifestation of the functional presence of the nucleic acid within a cell, including both transient expression and stable expression.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof For example, conservative amino acid changes may be made which, although they alter the primary sequence of the protein or peptide, do not normally alter its function. Conservative amino acid substitutions typically include substitutions within the following groups: glycine, alanine; valine, isoleucine, leucine; aspartic acid, glutamic acid; asparagine, glutamine; serine, threonine; lysine, arginine; phenylalanine, tyrosine. Modifications (which do not normally alter primary sequence) include in vivo, or in vitro chemical derivatization of polypeptides, e.g., acetylation, or carboxylation. Also included are posttranslational modifications; glycosylation, e.g., those made by modifying the glycosylation patterns of a polypeptide during its synthesis and processing or in further processing steps; e.g., by exposing the polypeptide to enzymes which affect glycosylation, e.g., glycosylation enzymes such as glycosyltransferases or glycosidases. Also embraced are sequences, which have phosphorylated amino acid residues, e.g., phosphotyrosine, phosphoserine, or phosphothreonine.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment. The isolated polypeptide also encompass a protein in a mixture, proteins expressed in cells and a component of a pharmaceutical composition.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the correspondirig positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, 20, and 25 amino acids. in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′ , Fv, F(ab′)₂, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Intracellular Antibodies: Research and Disease Applications, (Marasco, ed., Springer-Verlag New York, Inc., 1998), the disclosure of which is incorporated herein by reference in its entirety).

As used herein, antibodies can be produced by any known technique, including harvest from cell culture of native B lymphocytes, harvest from culture of hybridomas, recombinant expression systems and phage display.

The term “non-peptide analog” refers to a compound with properties that are analogous to those of a reference polypeptide. A non-peptide compound may also be termed a “peptide mimetic” or a “peptidomimetic”. See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the invention may be used to produce an equivalent effect and are therefore envisioned to be part of the invention.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

Amino acid substitutions can include those which: (1) reduce susceptibility to proteolysis, (2) reduce susceptibility to oxidation, (3) alter binding affnity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate; ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) In a preferred embodiment, a homologous protein is one that exhibits at least 80% sequence homology to the wild type protein, more preferred is at least 85% sequence homology. Even more preferred are homologous proteins that exhibit at least 86-90% sequence homology to the wild type protein. In a yet more preferred embodiment, a homologous protein exhibits at least 95%, 98%, 99% or 99.9% sequence identity. As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272(1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are:
Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (herein incorporated by reference). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

As used herein, the term “mutation” refers to any change of the DNA sequence within a gene or chromosome. Types of mutations include base substitution point mutations (e.g., transitions or transversions), deletions, and insertions. Missense mutations introduce a different amino acid into the sequence of the encoded protein; nonsense mutations introduce a new stop codon. For insertions or deletions, mutations can be in-frame (not changing the frame of the overall sequence) or frame shift mutations, which may result in the misreading of a large number of codons and often leads to abnormal termination of the encoded product due to the presence of a stop codon in the alternative frame.

As used herein, the term “effective amount” in reference to a pharmaceutical composition (Example 4) is an amount at least sufficient to treat, reduce, reverse or otherwise inhibit the given disease state. In the case of HIV-1 , this means an amount sufficient to reduce, reverse or otherwise inhibit HIV-1 replication. Similarly, for HIV-2, this means an amount sufficient to reduce, reverse or otherwise inhibit HIV-2 replication.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Throughout this specification and claims, the word “comprise” or variations such as “comprises” or “comprising”, will be understood to imply the inclusion of a stated integer or group of integers but not the exclusion of any other integer or group of integers.

### Polypeptides and Nucleic Acids Encoding APOBEC3G mutants

Provided herein are recombinant nucleic acid molecules capable of inhibiting replication of HIV-1 and/or HIV-2. The present invention provides recombinant nucleic acid molecules encoding APOBEC3G having at least one amino acid substitution at position 129. For example, the nucleic acid molecules include, e.g., SEQ ID NOs: 1, 3, 5 and 7 and related polypeptide sequences e.g., SEQ ID NOs: 2, 4, 6, and 8, analogs, variants, fragments and fiusions thereof encoding the mutant APOBEC3G conferring reduction or resistance to proteosomal degradation induced by HIV-Vif. The fuill-length nucleic acid sequence for the mutant P129D, which encodes the protein has been identified and sequenced (SEQ ID NO:1). The encoded amino acid sequence is also set forth SEQ ID NO:2. The APOBEC3G mutant of the present invention and other APOBEC3G mutants were generated as described in Example 1.

In addition, the full-length nucleic acid sequence for the mutant APOBEC3G P129G, which encodes the protein has been identified and sequenced (SEQ ID NO:3). The encoded amino acid sequence is also set forth SEQ ID NO:4. The mutation on position 129 of APOBEC3G protein inhibits HIV-1 replication in the presence of HIV-1 encoded viral protein, Vif.

Also provided herein is a full-length nucleic acid sequence for the mutant APOBEC3G P129A, which encodes the protein has been identified and sequenced (SEQ ID NO:5). The encoded amino acid sequence is also set forth SEQ ID NO:6. The mutation on position 129 of APOBEC3G protein inhibits HIV-1 replication in the presence of HIV-1 encoded viral protein, Vif. Additionally, the mutant APOBEC3G protein inhibits HIV-2 replication in the presence of HIV-2 encoded viral protein, Vif. The P129A mutant is an efficient inhibitor of HIV-1 or HIV-2 replication.

Another mutant exhibiting resistance to degradation caused by HIV-1 or HIV-2 Vifs is set forth as SEQ ID NO:7. The encoded amino acid sequence is also set forth SEQ ID NO:8. The P129F mutant protein inhibits HIV-1 or HIV-2 replication.

In one embodiment, the mutant APOBEC3G polypeptides retain deoxycytidine deaminase activity. The APOBEC3G protein of the present invention is, therefore, particularly useful in at least reducing HIV-1 and/or HIV-2 replication in cells. Preferably, the mutant protein inhibits HIV-1 and/or HIV-2 infection in humans.

The present invention also encompasses analogs of proteins or peptides of the present invention capable of neutralizing or inhibiting HIV-1 and/or HIV-2 infection. Analogs can differ from naturally occurring proteins or peptides by conservative amino acid sequence differences or by modifications which do not affect sequence, or by both. The invention includes analogs comprising a purified amino acid sequence sharing at least about 90% identity, preferably at least about 95%, more preferably at least about 99.9% identity with SEQID NOs:2, 4, 6 or 8 with the proviso that such analogs provide Vif inhibitory activity. In particular, a APOBEC3G polypeptide having at least an amino acid substitution at position 129 is within the scope of the invention.

Unlike wild type APOBEC3G, which contains Proline at position 129, the APOBEC3G mutants of the present invention comprise an amino acid substitution at position 129. The polypeptide of the present invention inhibits HIV-1 and/or HIV-2 replication in the presence of HIV-1 Vif or HIV-2 Vif while retaining its function. Preferably, the static state protein level of APOBEC3G mutant P129 D is not affected in the presence of HIV-1 Vif or HIV-2 Vif. In one embodiment, the mutation on the APOBEC3G protein is a P129D substitution. In another embodiment, the mutation on the APOBEC3G protein is a P129G substitution. In a preferred embodiment, the mutation on the APOBEC3G protein is a P129F substitution. In a more preferred embodiment, the mutation on the APOBEC3G protein is a P129A substitution. In yet another embodiment, the mutation on position AA129 may be any amino acid substitution except Proline. In some embodiments, it is contemplated that other APOBEC3G proteins having an amino acid substitution at 129 reduce or inhibit viral infectivity and replication in a host cell or subject.

Also included are polypeptides, which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

In addition to substantially full length-polypeptides, the present invention provides for enzymatically active fragments of the polypeptides. A polypeptide may be monomeric or polymeric. Further, a polypeptide may comprise a number of different domains each of which has one or more distinct activities.

In another aspect of the invention, provided herein is an recombinant nucleic acid molecule having a nucleic acid sequence comprising or consisting of mutant APOBEC3G sequence (SEQ ID NO: 1, 3, 5 or 7), analogs, homologs, variants and derivatives thereof. The invention also provides a nucleic acid molecule comprising or consisting of a sequence which is a degenerate variant of the mutant APOBEC3G gene. In a further embodiment, the invention provides a nucleic acid molecule comprising or consisting of a sequence which is a variant of the mutant APOBEC3G gene having at least 90% identity to the wild-type gene. The nucleic acid sequence can preferably have at least 91-95% identity to the wild-type gene. Even more preferably, the nucleic acid sequence can have 96%, 97%, 98%, 99%, 99.9% or even higher identity to the wild-type gene.

It will be readily apparent to a skilled artisan that the recombinant nucleic acid molecules may be expressed various host cells including mammalian cells, yeast cells, fungal cells, insect cells and bacterial cells. Accordingly, the nucleic acid molecules may also be codon optimized in which one or more amino acid sequence is changed, including a conservative amino acid substitution, addition, deletion or combination thereof.

The invention also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

The polypeptides and the polynucleotides of the present invention can be produced by chemical synthesis or, more commonly, by the artificial manipulation of isolated segments of nucleic acids, known genetic engineering techniques, particularly when construction expression vectors. Commercially available peptide synthesizers can be used in accordance with known protocols. Chemical synthesis of peptides is described in: S. B. H. Kent, Biomedical Polymers, eds. Goldberg and Nakajima, Academic Press, New York, pp. 2 13-242, 1980; Mitchell et al., J. Org. Chem., 43:2845-52, 1978; Tam et al., Tet. Letters, 4033-6, 1979; Mojsov et al., J. Org. Chem., 45:555-60, 1980; Tam et al., Tet. Letters, 2851-4, 1981; and Kent et al., Proceedings of the IV International Symposium on Methods of Protein Sequence Analysis, (Brookhaven Press, Brookhaven, N.Y., 1981.

The nucleic acid sequence fragments of the present invention display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hybridization to target sequences can be detected and optionally quantified. One of skill in the art will appreciate that the nucleic acid fragments of the present invention may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1) (suppl): 1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1) (suppl):1-60 (1999); Microarray Biochip: Tools and Technology, Schena (ed.), Eaton Publishing Company/BioTechniques Books Division (2000) (ISBN: 1881299376), the disclosures of each of which is incorporated herein by reference in its entirety.

### Antibodies to Mutant APOBEC3G Polypeptides

In another aspect of the invention, the invention provides isolated antibodies, including fragments and derivatives thereof, that bind specifically to the isolated polypeptides and polypeptide fragments of the present invention or to one or more of the polypeptides encoded by the isolated nucleic acids of the present invention. The antibodies of the present invention may be specific for linear epitopes, discontinuous epitopes or conformational epitopes of such polypeptides or polypeptide fragments, either as present on the polypeptide in its native conformation or, in some cases, as present on the polypeptides as denatured, as, e.g., by solubilization in SDS. Among the useful antibody fragments provided by the instant invention are Fab, Fab′, Fv, F(ab′)₂, and single chain Fv (SCFvs) fragments. These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) F(ab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine.

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies of the present invention will discriminate over adventitious binding to unrelated polypeptides by at least two-fold, more typically by at least 5-fold, typically by more than 10-fold, 25-fold, 50-fold, 75-fold, and often by more than 100-fold, and on occasion by more than 500-fold or 1000-fold.

Typically, the affinity or avidity of an antibody (or antibody multimer, as in the case of an IgM pentamer) of the present invention for a polypeptide or polypeptide fragment of the present invention will be at least about 1×10⁻⁶ M, typically at least about 5×10⁻⁷ M, usefully at least about 1×10⁻⁷ M, with affinities and avidities of 1×10⁻⁸ M, 5×10^{-9 M,} 1×10⁻¹⁰ M and even stronger proving especially useful.

The isolated antibodies of the present invention may be naturally-occurring forms, such as IgG, IgM, IgD, IgE, and IgA, from any mammalian species. For example, antibodies are usefully obtained from species including rodents—typically mouse, but also rat, guinea pig, and hamster—lagomorphs, typically rabbits, and also larger mammals, such as sheep, goats, cows, and horses. The animal is typically affirmatively immunized, according to standard immunization protocols, with the polypeptide or polypeptide fragment of the present invention.

Virtually all fragments of 8 or more contiguous amino acids of the polypeptides of the present invention may be used effectively as immunogens when conjugated to a carrier, typically a protein such as bovine thyroglobulin, keyhole limpet hemocyanin, or bovine serum albumin, conveniently using a bifunctional linker. Immunogenicity may also be conferred by fusion of the polypeptide and polypeptide fragments of the present invention to other moieties. For example, peptides of the present invention can be produced by solid phase synthesis on a branched polylysine core matrix; these multiple antigenic peptides (MAPs) provide high purity, increased avidity, accurate chemical definition and improved safety in vaccine development. See, e.g., Tam et al., Proc. Natl. Acad. Sci. USA 85:5409-5413 (1988); Posnett et al., J Biol. Chem. 263, 1719-1725 (1988).

Protocols for immunization are well-established in the art. Such protocols often include multiple immunizations, either with or without adjuvants such as Freund's complete adjuvant and Freund's incomplete adjuvant. Antibodies of the present invention may be polyclonal or monoclonal, with polyclonal antibodies having certain advantages in immunohistochemical detection of the proteins of the present invention and monoclonal antibodies having advantages in identifying and distinguishing particular epitopes of the proteins of the present invention. Following immunization, the antibodies of the present invention may be produced using any art-accepted technique. Host cells for recombinant antibody production—either whole antibodies, antibody fragments, or antibody derivatives—can be prokaryotic or eukaryotic. Prokaryotic hosts are particularly useful for producing phage displayed antibodies, as is well known in the art. Eukaryotic cells, including mammalian, insect, plant and fungal cells, are also useful for expression of the antibodies, antibody fragments, and antibody derivatives of the present invention. Antibodies of the present invention can also be prepared by cell free translation.

The isolated antibodies of the present invention, including fragments and derivatives thereof, can usefully be labeled. It is, therefore, another aspect of the present invention to provide labeled antibodies that bind specifically to one or more of the polypeptides and polypeptide fragments of the present invention. The choice of label depends, in part, upon the desired use. In some cases, the antibodies of the present invention may usefully be labeled with an enzyme. Alternatively, the antibodies may be labeled with colloidal gold or with a fluorophore. For secondary detection using labeled avidin, streptavidin, captavidin or neutravidin, the antibodies of the present invention may usefully be labeled with biotin. When the antibodies of the present invention are used, e.g., for Western blotting applications, they may usefully be labeled with radioisotopes, such as ³³P, ³²P, ³⁵S, ³H and ¹²⁵I. As would be understood, use of the labels described above is not restricted to any particular application.

Anti-X protein antibodies (for instance, antibodies that specifically recognize the APOBEC3G protein) may be produced using standard procedures described in a number of texts, including Harlow and Lane (Antibodies, A Laboratory Manual, CSHL, New York, 1988). The determination that a particular agent binds substantially only to the specified protein may readily be made by using or adapting routine procedures. One suitable in vitro assay makes use of the Western blotting procedure (described in many standard texts, including Harlow and Lane (Antibodies, A Laboratory Manual, CSHL, New York, 1988)). Western blotting may be used to determine that a given protein binding agent, such as an anti-X protein monoclonal antibody, binds substantially only to the X protein.

### Expression of Recombinant Mutant APOBEC3G Polypeptides

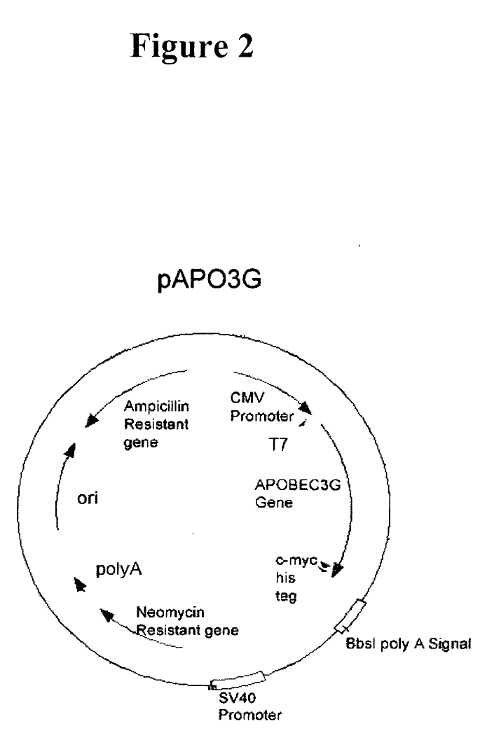

Proteins of the present invention are produced by culturing a recombinant host cell with an expression vector comprising a nucleic acid encoding the protein (FIG. 2), under the appropriate conditions to induce or cause expression of the protein. The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction.

Appropriate host cells include yeast, bacteria, archaebacteria, fungi, and insect and animal cells, including mammalian cells. Of particular interest are Drosophila melanogaster cells, Pichia pastoris and P. methanolica, Saccharomyces cerevisiae, E. coli, Bacillus subtilis, SF9 cells, SF21 cells, C129 cells, Saos-2 cells, Hi-cells, 293 cells, 293T cells, Neurospora, BHK, CHO, COS, and HeLa cells. Of greatest interest are A549, HeLa, HUVEC, Jurkat, BJAB, CHMC, and cell lines derived from T cells or macrophage.

In one embodiment, the proteins are recombinantly expressed in mammalian cells, especially human cells. Mammalian expression systems are also known in the art, and include retroviral systems. A mammalian promoter (i.e., a promoter functional in a mammalian cell) is any DNA sequence capable of binding mammalian RNA polymerase and initiating the downstream (3′) transcription of a coding sequence for a protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mNRA is formed by site-specific post-translational cleavage and polyadenylation. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, Example 2 describes a 293T cell transfected with the nucleic acid sequence have integrated the sequence e.g., into the host genome, at a selected location by homologous recombination between host and recombinant nucleic acid sequences. The sequence of the present invention may be preferably linked to one or more expression control sequences, so that the protein encoded by the sequence can be expressed under appropriate conditions in host cells that contain the isolated nucleic acid molecule. Stable genetic integration can be achieved in mammalian cells by biochemical selection, e.g., neomycin (Southern and Berg, 1982, J. Mol. Appl. Genet. 1:327-41) and mycophoenolic acid (Mulligan and Berg, 1981, Proc. Natl. Acad. Sci. USA: 78:2072-6).

Methods of introducing exogenous nucleic acid into hosts cells are well known in the art, and will vary with the host cell. Transfection of DNA into eukaryotic, such as human or other mammalian cells is an established technique in the art. The vectors are introduced into the recipient host cells as pure DNA (transfection) by, for example, precipitation with calcium phosphate (Graham and vander Eb, 1973, Virology 52:466) strontium phosphate (Brash et al., 1987, Mol. Cell Biol. 7:2013), electroporation (Neumann et al, 1982, EMBO J. 1:841), lipofection (Feigner et al., 1987, Proc. Natl. Acad. Sci USA 84:7413), DEAR dextran (McCuthan et al., 1968, J. Natl. Cancer Inst. 41:351), microinjection (Mueller et al., 1978, Cell 15:579), protopiast fusion (Schaiher, 1980, Proc. Natl. Acad. Sci. USA 77:2163-7), or pellet guns (Klein et al., 1987, Nature 327:70). Other methods include introducing the cDNA by infection with virus vectors, such as retroviruses (Bernstein et al., 1985, Gen. Engrg. 7:235) such as adenoviruses (Ahmad et al., J. Virol. 57:267, 1986) or Herpes (Spaete et al., Cell 30:295, 1982).

### Methods for Resisting Proteosomal Degradation induced by Vif Using Mutant APOBEC3G Polypeptide

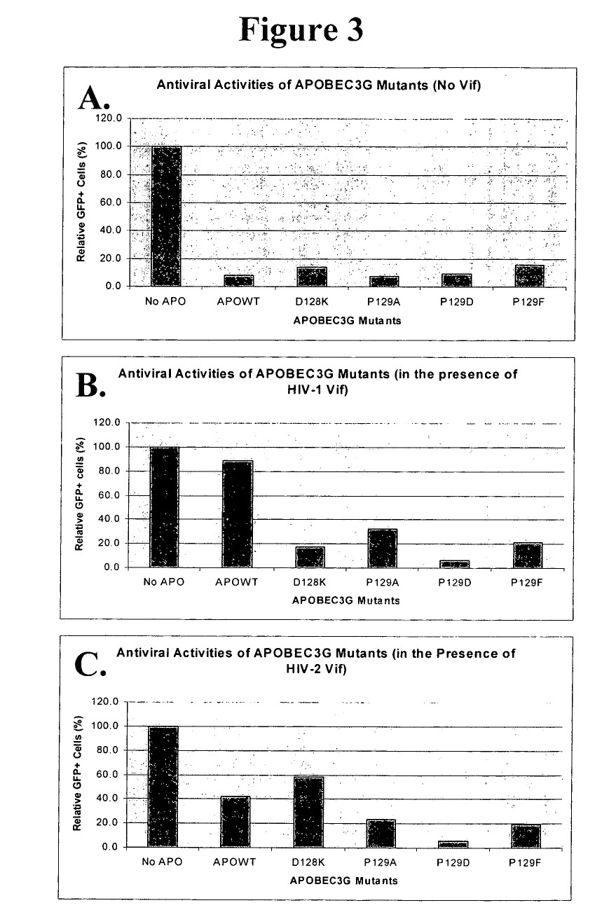

The mutant APOBEC3G protein of the present invention shows a viral inhibitory function in the presence of HIV-1 Vif or HIV-2 Vif similar to the wildtype APOBEC3G in absence of Vif (FIG. 3A). In particular, the P129D APOBEC3G mutant protein exhibits higher antiretroviral function compared to other APOBEC3G mutants (FIG. 3B, 3C). The effect of a single amino acid substitution mutation in APOBEC3G (e.g., P129D) on antiviral activity was determined in the absence and presence of HIV-1 Vif or HIV-2 Vif. Table I summarizes viral inhibition of the mutants tested.

TABLE I

Inhibition

| Mutant | HIV-1 (%) | HIV-2 (%) |
|---|---|---|
| 128K | 83 | 42 |
| 129D | 94 | 95 |
| 129F | 79 | 81 |
| 129A | 68 | 77 |

In one embodiment, the P129D protein inhibits about 90% viral activity in the presence of HIV-Vif. Preferably, the inhibition is about 94% for HIV-1 and 95% for HIV-2. More preferably the inhibition is 95-99.9% or greater. FIG. 3 shows the anti-retroviral activities of wild type APOBEC3G proteins and its mutants in the absence of HIV Vifs or in the presence of HIV-1 or HIV-2 (HIV-2Rod) Vifs. In the absence of HIV-1 or HIV-2 Vifs, all the mutants of APOBEC3G and the wild type protein that were shown in FIG. 3A could effectively inhibit HIV replication. In the presence of HIV-1 vif, wild type APOBEC3G lost its anti-retroviral activity, while the D128K, P129A, P129D and P129F still could inhibit HIV-1 effectively. Among these mutants, the P129D has the strongest resistance to HIV-1 Vifs. The other mutants are only partially effective in the presence of the Vifs. In the presence of HIV-2 vif, wild type APOBEC3G and D128K mutant partially lost their anti-retroviral activities, but the P129A, P129D and P129F mutants could inhibit viral replication efficiently. Again, the P129D has the strongest resistance to HIV-2 Vifs. While several APOBEC3G mutants inhibited HDV-EGFP replication in the presence of HIV-1 Vif or HIV-2 Vif and exhibited Vif-resistance in comparison to other mutants the results shown, however, indicate that the P129D APOBEC3G mutant is more efficient than the other mutants D128K, P129A or P129F. Accordingly, the P129D APOBEC3G mutant has the strongest resistance to HIV-1 and HIV-2 Vifs, thereby, inhibiting viral replication more efficiently than other APOBEC3G mutants.

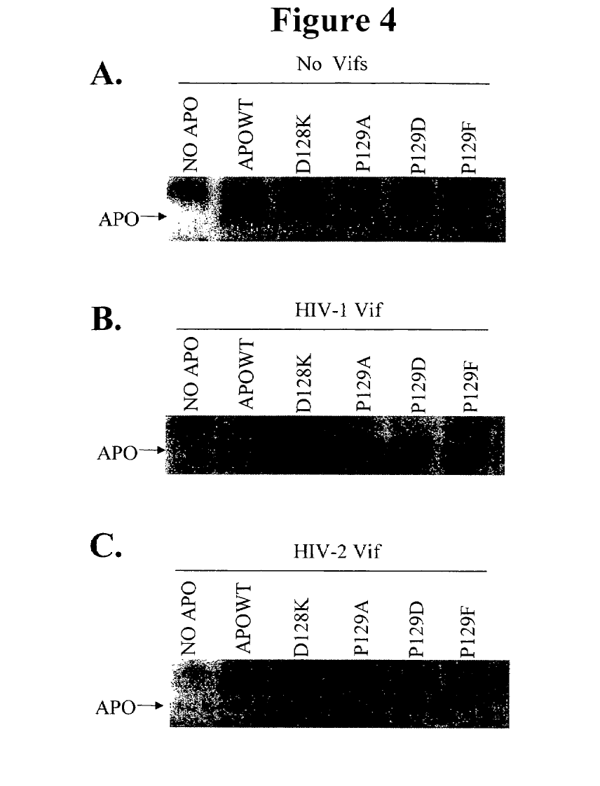

There are several advantages to using the APOBEC3G of the present invention. HIV-1 Vif or HIV-2 does not reduce intracellular steady-state levels of the APOBEC3G mutant, a salient feature of the invention. The P129D mutant displayed the Vif-resistant phenotype. Using the methods as described in Example 2, the APOBEC3G mutant's antiviral activity was determined in the presence and absence of HIV-1 or HIV-2 Vif. FIG. 4 showed P129D is resistant to both HIV-1 Vif and HIV-2 Vif. APOBEC3G expression vectors (pcDNA-APO3G (FIG. 2) and pAPO3G-P129D) and Vif expressing vector (pC-Help) were co-transfected into 293T cells. The cell lysates were harvested 48 hours after transfection, and subject to electrophoresis on SDS-page and immunoblotting analysis (Example 3). The APOBEC3G was detected by anti-cMyc antibody. Result showed that the amount of wild type APOBEC3G protein was reduced in the presence of HIV-1 (vif+), but the amount of D128K, P129A, P129D and P129F mutant protein were not reduced when the Vif was co-expressed. When HIV-2 was co-expressed with the APOBEC3G and its mutants in viral producing cells, only P129D was resistant to the degradation induced by HIV-2 Vif (FIG. 4C).

In another aspect of the invention, a method is provided for reducing, inhibiting, suppressing or preventing HIV-1 and/or HIV-2 infection in host cells by resisting, inhibiting or reducing proteosomal degradation induced by Vif comprising administering a therapeutically effective amount of the mutant APOBEC3G polypeptide (Example 4). In one embodiment, the method provides mutant APOBEC3G that still retains deoxycytidine deaminase activity. By administering the mutant APOBEC3G as an anti-viral compound, HIV-1 and/or HIV-2 infection is at least decreased. In one embodiment, the mutant APOBEC3G is administered in combination with other anti-viral drugs including interleukin, antibiotics, protease inhibitors, non-nucleoside reverse-transcriptase inhibitors. In other embodiments, the method provides administering the polypeptide of the present invention as part of a cellular immune defense mechanism against Vif.

As is well known in the art, viral infectivity assays show the inhibition on viral replication and viral infectivity can be measured in various ways. For example, the mutant APOBEC3G can be, tested in the viral infectivity assay as described in (Example 5). Additional examples of viral infectivity are also known in the art. Other methods and techniques may also be suitable for the measurement of viral infectivity, as would be known by one of skill in the art.

The following examples are for illustrative purposes and are not intended to limit the scope of the invention.

### EXAMPLE 1

### Generation of Recombinant APOBEC3G Mutant Constructs

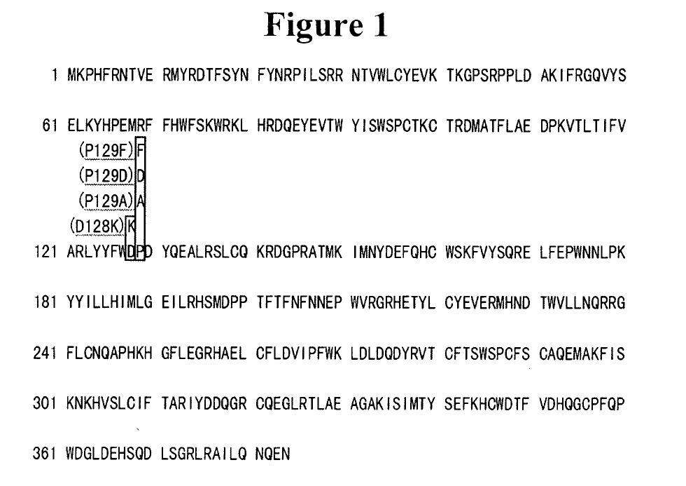

The mutations employed to generate APOBEC3G mutant constructs were done by using either the Multi-Site Mutagenesis Kit (Stratagene) or by PCR-based mutagenesis and was sequenced thereafter to verify the mutation. The amino acid substitutions were introduced into pcDNA-APO3G, Kao et al., J. Virol. 77, 11398-11407 (2003). From N-terminal of APOBEC3G to C-terminal, the following amino acid substitutions were made as shown in FIG. 1:

### D128K

### P129A

### P129G

### P129D

### P129F

The following plasmids have been described previously: pHDV-EGFP, Unutmaz et al., J. Exp. Med 189, 1735-1746(1999); pNL4-3, Adachi et al., J. Virol. 59, 284-291(1986); pcDNA-APO3G, Kao et al., J. Virol. 77, 11398-11407 (2003); pC-Help, Mochizuki et al., J. Virol. 72, 8873-8883(1998); pC-HelpΔVif, Kao et al. J. Virol. 77:11398-407, 2003; and pΔNC, Ott et al., J. Virol. 77, 5547-5556 (2003). The pcDNA expression vectors contain amino acid substitution at position 129 on APOBEC3G cDNA (Genbank AN BC024268). Transfections employed PolyFect reagent (Qiagen, Inc.) according to the manufacturer's instructions, with equimolar ratios of all plasmids and with harvests after 36 hours unless otherwise mentioned.

### EXAMPLE 2

### Host Cell Transfections, Infections, and Flow Cytometry Analysis

Wild type APOBEC3G and its mutants were cotransfected with pHDV-EGFP (HIV-1 producing vector), pCMV-g (VSVg envelope expression vector) and pcHelpΔvif (Vif− bars) or pcHelp (expressing HIV-1 vif, Vif+ bars) to 293T cells using a CalPhos Mammalian Transfection Kit (BD Biosciences). The viruses were harvested 48 hours after transfection by filtering through 0.45 mm syringe filter (Corning). The p24 capsid of each virus was measured by P24 ELISA kit (PerkinElmer), and viruses that equivalents to 30 ng of p24 from each sample were used to infected 293T cells in 6-well plates. The infected 293T cells were harvested 48 hours after infection, and subjected to FACScan analysis (Becton-Dickinson). The GFP positive cells in sample that transfected with pHDV-EGFP, pCMV-g, wild type APOBEC3G and pcHelp was set to 100%.

To deternine the effects of wild-type or mutant APOBEC3Gs on HIV-1 or HIV-2 replication in the absence of Vif (Vif−) the pHDV-EGFP, pC-HelpΔVif, pHCMV-G and pcDNA-APO3G or mutants of pcDNA-APO3G were cotransfected using 20:15:4:4 μg of DNA respectively. The molar ratios of pHDV-EGFP, pCHelpΔVif, pHCMV-G and the pcDNA-APO3G or APOBEC3G mutant plasmids were approximately 1:1:0.4:0.4, respectively.

To determine the effects of wild type or mutant APOBEC3Gs on HIV-1 or HIV-2 replication in the presence of Vif (Vif+) the pHDV-EGFP, pC-Help, pHCMV-G, and pcDNA-APO3G or mutants of pcDNA-APO3G were cotransfected using 20:15:4:4 μg of DNA, respectively. The molar ratios of pHDV-EGFP, pC-Help, and the pcDNA-APO3G wild type or mutant plasmids were approximately 1: 1:0.4:0.4, respectively.

To determine whether the mutants are dominant over the wild-type APOBEC3G, pHDV-EGFP, pC-Help, and pHCMV-G plasmids were cotransfected with pcDNA-APO3G and/or mutant APOBEC3G plasmid pAPO129D. The total amount of the APOBEC3G plasmids was 4 μg in each experiment, and the molar ratio of pHDV-EGFP, pC-Help, pHCMV-G, and APOBEC3G plasmids was 1:1:0.4:0.4.

The effects ofthe wild type and mutant APOBEC3G on HIV-1 replication in the presence of HIV-1 Vif are shown in FIG. 3B. The effect of the P129D mutant APOBEC3G in the presence of HIV-1 Vif expression showed dramatic reductions in GFP-positive cells to less than 10%.

The effects of the wild type and mutant APOBEC3G on HIV-2 replication in the presence of HIV-2 Vif are shown in FIG. 3C. Expression of P129A and P129F APOBEC3G mutants slightly decreased GFP expression levels in infected cells, however, the P129D APOBEC3G mutant showed marked resistance to HIV Vifs. The effect of the P129D mutant APOBEC3G in the presence of HIV-2 Vif expression showed dramatic reductions in GFP-positive cells to less than 10%.

### EXAMPLE 3

### APOBEC3G Mutants P129A, P129D and P129F Resist Proteosomal Degradation by HIV-1 and HIV-2 Vifs

Intracellular steady-state levels of wild-type APOBEC3G and mutant APOBEC3G proteins were determined by Western blotting detection of C-terminal myc-tagged APOBEC3G proteins in the presence and absence of HIV Vifs. For co-immunoprecipitation, an anti-c-Myc antibody (Sigma-Aldrich) was coupled to paramagnetic beads according to manufacturer's instructions (Dynal Biotech). 293T cells were cotransfected with APOBEC3G expressing plasmids and either pC-Help or pC-HelpΔVif. Approximately 36 hours after transfection, 2×10⁻⁶ cells were harvested, washed twice with ice-cold PBS, and lysed in 1 ml of cell extraction buffer (20 mM Tris-Cl, pH 8.0, 137 mM NaCl, 1 mM EDTA, 1 mM NaVO₃, 10% glycerol, 1% Triton X-100 and protease inhibitor cocktail [Roche]). Cell extracts were adjusted to equivalent protein concentration by using Bradford reagent (BioRad Laboratories), and equal aliquots were then used for co-immunoprecipitation and Western blotting analysis.

Cell extracts were centrifuiged at 1,500×g for four minutes, and the supernatants incubated with anti-c-Myc antibody conjugated paramagnectic beads for three hours in slow rotation on RKDynal rotor (Dynal Biotech) at 4° C. After incubation, the beads were washed three times with 50 mM Tris-HCl, pH 7.5, 500 mM LiCI, 1 mM NaVO₃ and 0.5% Triton X-100, 3 times with 50 mM Tris-HCI, pH 7.5, 500 mM LiCl, 1 mM NaVO₃, and once with 1 mM NaVO₃.

The bound proteins were eluted from the beads by heating to 90° C. for five minutes in SDS-PAGE loading buffer. For cell lysis, 2×10⁶ cells were harvested, washed with ice-cold PBS 36 hours after transfection,, lysed in 1×SDS-PAGE loading buffer, and heated to 90° C. for five minutes. For Western blot analysis, the myc epitope-tagged APOBEC3G proteins were detected by using the anti-c-Myc antibody and the HIV-1 Vif protein was detected by using anti-H1V-1HXB2 Vif antiserum (Dana Gabuzda, Dana-Farber Cancer Institute) obtained through the AIDS Reagents and Reference Program, Division of AIDS, NIAID, NIH.

The result in FIG. 4 showed that the amount of wild type APOBEC3G protein was reduced in the presence of HIV-1 (Panel B) but the amount of D128K, P129A, P129D and P129F APOBEC3G protein were not reduced. If HIV-2 Vif was co-expressed with the APOBEC3G and its mutants in viral producing cells, only P129D was resistant to the degradation induced by HIV-2 Vif (Panel C), P129F and P129A were partially resistant. The steady-state levels of wild-type APOBEC3G protein, but not the mutant APOBEC3G proteins, were significantly depleted in the presence of HIV-1 or HIV-2 Vif. Therefore, P129D confers better resistance to proteosomal degradation by either HIV-1 Vif or HIV-2 Vif.

### EXAMPLE 4

### Pharmaceutical Compositions

The mutant polypeptide composition in pharmaceutical formulations can be used to treat lentiviral infections; such as HIV and AIDS, by blocking replication of an immunodeficiency virus. Determining the effective amount of the instant pharmaceutical compositions can be done based on animal data using routine computational methods. A therapeutically effective amount of an agent can be administered in one or in multiple doses during a course of treatment. Compositions that include a therapeutic agent can be administered as needed.

In one embodiment, the effective amount contains between about 10 ng and about 100 μg of the instant nucleic acid molecules per kg body mass. In another embodiment, the effective amount contains between about 100 ng and about 10 μg of the nucleic acid molecules per kg body mass. In a further embodiment, the effective amount contains between about 1 μg and about 5 μg of the nucleic acid molecules per kg body mass. In another embodiment the effective amount contains between about 10 μg and about 100 μg of the nucleic acid molecules per kg body mass. In a preferred embodiment the effective amount contains between about 100 μg and 500 μg of the nucleic acid molecules per kg body mass. In another embodiment the effective amount contains between about 500 μg and 1000 μg of the nucleic acid molecules per kg body mass. In another embodiment the effective amount contains between about 1 mg and 2 mg of the nucleic acid molecules per kg body mass.

The compounds of the invention may also be admixed, encapsulated, conjugated or otherwise associated with other molecules, molecule structures or mixtures of compounds, as for example, liposomes, receptor targeted molecules, oral, rectal, topical, transdermal, intratracheal, inhalation or other formulations, for assisting in uptake, distribution and/or absorption. Representative United States patents that teach the preparation of such uptake, distribution and/or absorption assisting formulations include, but are not limited to: U.S. Pat. Nos. 5,354,934; 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference. Other mechanism for drug delivery such as sustained-release (e.g., polymer, microcapsules), mechanical apparatus including implantable infusion devices and pumps can also be administered.

The effective amount of pharmaceutical composition can be administered to a human patient in need from 1-8 or more times daily or every other day. Dosage is dependent on severity and responsiveness of the effects of the disease to be treated, with a course of treatment lasting from several days to months or until a cure is effected or a reduction of the effects is achieved. The actual effective amount, or dosage, administered may take into account the size and weight of the patient, whether the nature of the treatment is prophylactic, therapeutic in nature, the age, weight, health and sex of the patient, the route of administration, and other factors including as prescribed by a clinician.

Pharmaceutical compositions may also contain suitable excipients and auxiliaries which facilitate processing of the nucleic acids into preparations which can be used pharmaceutically. Generally, agents for use in the invention are formulated in either parenteral or enteral forms, usually enteral formulations, more particularly oral formulations. Preferably, the agents for use in the invention are formulated for parenteral administration, e.g., by subcutaneous, intradermal, intraperitoneal, intravenous, or intramuscular injection. Additionally, suitable solutions for administration parenterally or orally, and compositions which can be administered bucally or sublingually, including inclusion compounds; contain from about 0.1 to about 99 percent by weight of active ingredients, together with the excipient.

The pharmaceutical preparations of the present invention are manufactured in a manner which is itself well known in the art. For example, the pharmaceutical preparations may be made by means of conventional mixing, granulating, dragee-making, dissolving, or lyophilizing processes. The process to be used will depend ultimately on the physical properties of the active ingredient used.

### EXAMPLE 5

### Viral Infectivity Assay

Cell-based assays, membrane vesicle-based assays and membrane fraction-based assays can be used to identify compounds that influence interactions in the Vif-mediated APOBEC3G degradation pathway. Cell lines that express a Vif or APOBEC3G, or a fusion protein containing a domain or fragment of such protein (or a combination thereof), or cell lines (e.g., COS cells, CHO cells, HEK293 cells, etc.) that have been genetically engineered to express such protein(s) or fusion protein(s) (e.g., by transfection or transduction of APOBEC3G or Vif DNA) can be used. Test compound(s) that influence the degradation pathway for example, can be detected by monitoring a change in the level or amount or turnover rate of APOBEC3G or a fusion protein containing a domain or fragment thereof.

For cell-based assays, about 20,000 to 250,000 cells are infected with the desired pathogen, such as HIV-1 , and the incubation continued for 3-7 days. The test agent can be applied to the cells before, during, or after infection with the virus. The amount of virus and agent administered can be determined by skilled artisan. In some cases, several different doses of the potential therapeutic agent can be administered to identify optimal dose ranges. Following transfection, assays are conducted to determine the resistance of the cells to infection by various agents.

For example, the presence of an H1V-1 antigen can be determined by using antibody specific for an HIV-1 protein then detecting the antibody. Examples of HIV-1 antibodies include antibody against p24 of HIV in the ELISA kit (Perkin-Elmer) and anti-H1V-HXB2 Vif antiserum against HIV-1 Vif protein (Dana Gabuzda, Dana-Farber Cancer Institute) obtained through the AIDS Reagents and Reference Program, Division of AIDS, NIAID, NIH. In one example, the antibody that specifically binds to an HIV-1 protein is labeled, for example with a detectable marker such as a flurophore. Alternatively, the antibody is detected by using a secondary antibody containing a label. The presence of bound antibody is then detected, for example using microscopy, flow cytometry, and ELISA.

An assay can be designed so as to be useful in high-throughput assays. The cells can be cultured in a suitable receptacle, preferably in a receptacle used for high throughput screening (e.g., a multi-well plate).

### SEQUENCE LISTING

SEQ ID NO:1 P129D nucleic acid
SEQ ID NO:2 P129D AA
SEQ ID NO:3 P129G nucleic acid
SEQ ID NO:4 P129G AA
SEQ ID NO:5 P129A nucleic acid
SEQ ID NO:6 P129A AA
SEQ ID NO:7 P129F nucleic acid
SEQ ID NO:8 P129F AA
SEQ ID.NO:9 D128K nucleic acid
SEQ ID NO:10 D128K AA

What is claimed is:

1. An isolated polypeptide comprising or consisting of polypeptide sequences selected from the group consisting of encoded APOBEC3G proteins having at least one amino acid substitution at position 129 that is capable of resisting proteosomal degradation induced by HIV-1 and/or HIV-2 Vifs.

2. The polypeptide of claim 1 wherein the polypeptide sequence is selected from the group consisting of: SEQ ID NOs: 2, 4, 6, 8 and related polypeptide sequences such as analogs, variants, fragments and fusions thereof

3. The polypeptide of claim 1 wherein the polypeptide comprises at least one additional amino acid substitution.

4. The polypeptide of claim 1 wherein the polypeptide is used to contact a cell to reduce replication of HIV produced from said cell.

5. An isolated nucleic acid molecule comprising or consisting of a polynucleotide sequence encoding the polypeptide of claim 1.

6. The polypeptide of claim 1 wherein the polypeptide encodes a protein wherein said protein is used to contact a cell to reduce replication of HIV produced from said cell.

7. A vector comprising the nucleic acid molecule of claim 5.

8. A host cell comprising the nucleic acid molecule of claim 5.

9. A pharmaceutical composition comprising the polypeptide of claim 1.

10. A kit comprising the vector of claim 7.

11. A kit comprising the host cell of claim 8.

12. A kit comprising the pharmaceutical composition of claim 9.

13. A method for reducing HIV-1 and/or HIV-2 infection in a cell comprising administering to a subject therapeutically effective amount of the polypeptide of claim 1.

14. An isolated polypeptide comprising or consisting of a polypeptide sequence selected from the group consisting of:
(a) SEQ ID NO:2;
(b) SEQ ID NO:4;
(c) SEQ ID NO:6;
(d) SEQ ID NO:8;
(e) a polypeptide sequence at least 90%, at least 95%, at least 96%, at least 97%, at least 98%, at least 99% or at least 99.9% identical to SEQ ID NOs:2, 4, 6 or 8; and
(f) an analog, variant, fragment or fusion thereof
wherein said polypeptide is capable of resistance to proteosomal degradation induced by HIV-1 and/or HIV-2 Vifs.

15. The polypeptide of claim 14 wherein the polypeptide comprises at least one additional amino acid substitution.

16. The polypeptide of claim 14 wherein the polypeptide encodes a protein wherein said protein is used to contact a cell to reduce replication of HIV produced from said cell.

17. An isolated nucleic acid molecule comprising or consisting of a polynucleotide sequence encoding the polypeptide of claim 14.

18. The polypeptide of claim 14 wherein the polypeptide encodes a protein wherein said protein is used to contact a cell to reduce replication of HIV produced from said cell.

19. A vector comprising the nucleic acid molecule of claim 17.

20. A host cell comprising the nucleic acid molecule of claim 17.

21. A pharmaceutical composition comprising the polypeptide of claim 14.

22. A kit comprising the vector of claim 19.

23. A kit comprising the host cell of claim 20.

24. A kit comprising the pharmaceutical composition of claim 21.

25. A method for reducing HIV-1 infection in a cell comprising administering to a subject therapeutically effective amount of the polypeptide of claim 14.

26. An isolated nucleic acid molecule comprising or consisting of a polynucleotide selected from the group consisting of:
(a) SEQ ID NO:1;
(b) SEQ ID NO:3;
(c) SEQ ID NO:5;
(d) SEQ ID NO:7;
(e) a nucleic acid sequence that is a degenerate variant of (a), (b), (c) or (d);
(f) a nucleic acid sequence that encodes a polypeptide having the amino acid sequence of SEQ. ID NOs:2, 4, 6 or 8;
(g) a nucleic acid sequence that encodes a polypeptide at least 90%, at least 95%, at least 98%, at least 99% or at least 99.9% identical to SEQ ID NOs:1, 3, 5 or 7; and
(h) a nucleic acid sequence that hybridizes under stringent conditions to SEQ ID NO: 1,3, 5 or 7; and
wherein said polynucleotide that encodes a polypeptide, which is capable of resistance to proteosomal degradation induced by HIV-1 and/or HIV-2 Vifs.

27. A vector comprising the nucleic acid molecules of claim 26.

28. A host cell comprising the nucleic acid molecules of claim 26.

29. A pharmaceutical composition of claim 26.

30. A kit comprising the vector of claim 27.

31. A kit comprising the host cell of claim 28.

32. A kit comprising the pharmaceutical composition of claim 29.

33. A method for reducing or inhibiting HIV-1 and/or HIV-2 replication in a cell infected with HIV-1 and/or HIV-2 by contacting the cell with an effective amount of a polypeptide selected from the group consisting of:
(a) SEQ ID NO:2;
(b) SEQ ID NO:4;
(c) SEQ ID NO:6
(d) SEQ ID NO:8
(e) a polypeptide sequence at least 90%, at least 95%, at least 96%, at least 97%, at least 98%, at least 99.9% identical to SEQ ID NOs:2, 4, 6 or 8; and
(f) an analog variant, fragment or fusion thereof
wherein said polypeptide is capable of resistance to proteosomal degradation induced by HIV-Vif.

34. An isolated antibody or antigen-binding fragment or derivative thereof which binds selectively to the isolated polypeptide of claims 1, 14, 26 or 33.
